(19) United States
(12) Patent Application Publication
(10) Pub. No.: US 2004/0151739 A1
(43) Pub. Date: Aug. 5, 2004

(54) Use of a composition for the stimulation of nerve growth, the inhibition of scar tissue formation, the reduction of secondary damage and/or the accumulation of macrophages

(76) Inventors: Philippe P. Monnier, Tubingen (DE); Bernhard K. Mueller, Tubingen (DE); Jan Schwab, Tubingen (DE)

Correspondence Address: CLARK & ELBING LLP, 101 FEDERAL STREET, BOSTON, MA, 02110, US

(21) Appl. No.: 10/451,487

(22) PCT Filed: Dec. 20, 2001
(86) PCT No.: PCT/EP01/15147

(30) Foreign Application Priority Data
Dec. 22, 2000 (DE) 10064195.4

(51) Int. Cl. A61K 39/08

(52) U.S. Cl. 424/239.1; 514/12; 530/350

(57) ABSTRACT

The invention relates to the use of a composition, comprising a fusion protein and at least one transporter for the in-vivo inhibition of scar tissue formation, the in-vivo reduction of secondary damage and/or the in-vivo accumulation of macrophages. The fusion protein contains at least one binding domain for the transporter and at least one modulation domain for the covalent modification of small GTP-binding proteins. The transporter permits the uptake of the fusion protein in a target cell.

The present invention relates to the use of a composition comprising a fusion protein and at least one transporter for the in vivo stimulation of nerve growth, the in vivo inhibition of scar tissue formation, the in vivo reduction of secondary damage and/or the in vivo accumulation of macrophages, where the fusion protein contains at least one binding domain for the transporter and at least one modulation domain for the covalent modification of small GTP-binding proteins, and where the transporter ensures the uptake of the fusion protein in a target cell.

The spinal cord and the brain form the central nervous system (CNS) in vertebrates. The spinal cord extends along the longitudinal axis of the body and is surrounded by the spinal canal. In human beings, the spinal cord is divided into eight cervical segments, twelve thoracic segments, five lumbar segments, five sacral segments and one or two coccygeal segments. The central gray substance, with its lateral projections (the anterior horn and the posterior horn), is formed by the cytosomes of the nerve cells, while the peripheral white substance is formed by the medullated nerve fiber bundles. The afferent (ascending or sensory) neural pathways and efferent (descending or effector) neural pathways run in the white substance. The efferent pathways in the spinal cord are either pyramidal (for voluntary movements) or extrapyramidal (for involuntary movements and for the distribution of the muscular tone). The majority of the pyramidal fibers run with a cross-over in the lateral pyramidal tract of the opposite side, and to a smaller extent without a cross-over in the anterior pyramidal tract to the cells in the anterior horn and the posterior horn in the various segments of the spinal cord.

The spinal cord and the brain are formed by cells of two types: the nerve cells or neurons and glial cells. The glial cells can be either oligodendrocytes or astroytes. The oligodendrocytes form the myelin sheath of the nerve axons, while the astrocytes supply the nerve cells or neurons with nourishment, absorb the neurotransmitters secreted, and form the blood-brain barrier. Myelin is the fatty insulating sheath that surrounds the nerves in a helical form. This coating ensures the trouble-free conduction of electrical impulses along the nerve.

The myelin sheath is attacked and destroyed in numerous diseases, such as: multiple sclerosis, encephalitis periaxialis, diffuse sclerosis, acute disseminated encephalomyelitis, neuromyelitis optica, SMON (subacute myelo-optical neuropathy), congenital demyelinization disorders (such as leukodystrophy), and the generally immune-mediated inflammatory diseases of the nervous system, such as neurologic Behcet syndrome and Kawasaki syndrome. This damage leads to an electrical conduction blockade and neurologic symptoms, with the loss of numerous important functions. Injury to the spinal cord, e.g. as a result of an accident, leads to a lasting abolition of the conduction function of the nerve fibers affected. Paralysis caused by the complete abolition of at least one segment is called transverse lesion of the spinal cord with paraplegia. This means the loss of sensory functions (e.g. temperature, pain or pressure sensations), motor functions (voluntary and involuntary movements) and vegetative functions (e.g. bladder and intestinal function) for all areas that lie under the affected segment. Owing to the poor regenerative capability of the nerve fibers, the paralysis of the voluntary movements and the complete loss of sensation are permanent.

Injury-induced CNS lesions lead to the death of the cells at the site of the injury itself. This is accompanied by the formation of large amounts of regeneration-inhibiting cell residues and inhibitory myelin components. These are rapidly removed by macrophages in the case of injuries to the peripheral nervous system. In the CNS, this inflammatory reaction is delayed and is less intense. As a result, the inhibitory myelin residues persist at the site of the injury for a long time. If macrophages activated by contact with peripheral nerves are introduced into the site of injury to the CNS, they remove the myelin residues and thereby induce a regeneration of the neural axons [see O. Lazarov-Spiegler, A. S. Solomon and M. Schwarz, Glia, 24, (1998), 329-337].

The primary lesion caused by mechanical means (primary damage) is aggravated by secondary phenomena (secondary damage), and scar formation (cicatrization) begins. To this is added a spatially extensive reaction of the astrocytes. This is indicated by an increase in the presence of the glial fibrillary acidic protein (GFAP). The astrocytes that are near the site of the injury raise the production of vimentin and nestin, and cell division is observed. A new glia, glia limitans, is formed at the boundary between the meningeal cells that have migrated in and the surviving astrocytes. As a result, the astrocytes become hypertrophic (they increase in size) in this region, they put out numerous fine filaments and some of them divide.

The finished scar mainly consists of hyperfilamentous astrocytes whose filaments interweave with one another across many gap junctions and tight junctions, and they are so tightly packed that only a small extracellular space is left free. The scar therefore forms a virtually insurmountable mechanical obstacle to the regenerating axons. In addition, the scar also contains numerous substances that inhibit regeneration. These are mainly chondroitin sulfate proteoglycans (CSPGs, e.g. aggrecan, versican, neurocan, brevican, phosphacan and NG2) and tenascin [see J. W. Fawcett and R. A. Asher, Brain Research Bulletin, 49 (1999), 377-391].

However, there are also some neurologic and neurodegenerative diseases of the peripheral and central nervous system in which the neurons perish. Examples of these are Alzheimer's disease, Parkinson's disease, multiple sclerosis and similar disorders where nerve fibers are lost and demedullated, as well as amyotrophic lateral sclerosis and other motor neuron diseases, ischemia, stroke, epilepsy, Huntington's disease, the AIDS-dementia complex and prion diseases.

The aim of the present investigations was therefore to effect the regeneration of the nerve axons in the injured region in the case of lesions to the spinal cord, and to stimulate nerve growth in other diseases of the peripheral and central nervous system. The formation of scar tissue in the central nervous system of mammals represents an enormous obstacle to the regeneration of growing nerve fibers. For this reason, the slowing-down or inhibition of cicatrization and the stimulation of the growth of nerve fibers are essential therapeutic objectives in the field of neuroregenerative therapeutic concepts.

The way the signal transduction paths in the nerve cells can be influenced offers a starting point. It is well known that the activation of small GTP-binding proteins belonging to the group of Rho GTPases (Rho A, B, C) leads to an intense growth inhibition of the nerve fibers under the conditions of cell cultures [see B. K. Mueller, Annu. Rev. Neurosci., 22 (1999), 351-388]. Experimental evidence indicates that the activatin of Rho A, B and/or C inside the nerve fibers by powerful regeneration-inhibiting proteins of the adult mammalian brain (NOGO, MAG, RGM, ephrin-AS), which proteins attack the outside of the membrane, represents an essential mechanism of the inhibition of nerve fiber growth [see Z. Jin and S. M. Strittmatter, J. Neurosci., 17 (1997), 6256-6263; also M. Lehmann, A. Fournier, I. Selles-Navarro, P. Dergham, A. Sebok, N. Leclerc, G. Tigyi, and L. McKerracher, J. Neurosci., 19 (1999), 7537-7547, and S. Wahl, H. Barth, T. Ciossek, K. Aktories and B. K. Mueller, J. Cell. Biol., 149 (2000) 263-270]. In the case of newly proliferating nerve fibers, the activation of Rho A-C leads to the collapse of the growth cone, the distal tip of the neurite, and so blocks the formation of new neural pathways.

In the case of mature oligodendrocytes, the binding of the nerve growth factor (NGF) on the p75 receptor leads to apoptosis [see P. Casaccia-Bonnefil, B. D. Carter, R. T. Dobrowsky and M. V. Chao, Nature, 383 (1996), 716-719]. In the case of neuronal cells, the intracellular domain of p75 is directly bound to Rho A-C. The binding of neurotrophins on the p75 receptor reduces the activity of Rho A and so leads to neurite elongation. If the activity of Rho A is permanently raised by a Val¹⁴-Rho A mutation, then the addition of NGF does not lead to neurite growth [see T. Yamashita, K. L. Tucker and Y.-A. Barde, Neuron, 24 (1999), 585-593]. The direct effect exerted on Rho A suppresses the signal transduction cascade of NGF-p75 and should therefore also inhibit the apoptotic action of the binding of NGF on p75 in the case of the oligodendrocytes.

It is known from the prior art that the bacterial exoenzyme C3-transferase is a specific inhibitor of Rho A, B and C [see K. Aktories, G. Schmidt and I. Just, Biol. Chem., 381 (2000), 421-426]. This protein ADP-ribosylates Rho A-C on argenine group 41 and so inhibits these Rho GTPases [see Aktories et al. (2000), quoted above]. To obtain a sufficient activation blockade of the Rho GTPases, over 90% of the intracellular Rho A-C proteins must be ADP-ribosylated and so inactivated. However, C3-transferase has a very low membrane permeability and is therefore absorbed by the cells only in very small amounts (about 1% of the initial amount). Consequently, very large amounts of C3-transferase are needed to inactivate 90% of the intracellular Rho A-C proteins. For this reason, very large amounts of these C3-transferases, which are known to be toxins, would have to be administered for pharmaceutical applications, and therefore toxic side effects could not be ruled out. The pharmacologic use of C3-transferase is therefore unsuitable on the grounds of toxicity alone.

To ensure a better transport of the active component, C3-transferase, through the plasma membrane of the cells, a chimeric fusion protein has been prepared [see German Patent No. 197 35 105]. The binary actin ADP-ribosylating C2 toxin from Clostridium botulinum was used for this purpose. C2 toxin consists of two proteins: the C2I component, which is enzymatically active, and the C2II component, which ensures the binding on the plasma membrane and a subsequent. translocation. The enzymatic activity of C2I protein is located in the C-terminal region, while the binding on C2II involves the N-terminal region. To introduce C3-transferase into the cells with the aid of this efficient uptake mechanism, a chimeric fusion protein has been prepared from C3-transferase (from Clostridium limosum) and from the N-terminal C2I protein (see FIG. 1). This C3-C2IN fusion protein is now introduced into the cells with the aid of the binding protein C2II [see H. Barth, C. Hofmann, C. Olenik, I. Just, and K. Aktories, Infect. Immun., 66 (1999), 1364-1369]. The complex formed from C3-C2IN and C2II is internalized via receptor-mediated endocytosis and reaches the intracellular vesicles. From these vesicles, the C3-C2IN fusion protein reaches the cytosol, where it can exert its effect and can ADP-ribosylate—and hence inactivate—Rho A-C. This binary protein complex formed from C3-C2IN and C2II, combines the functional properties of C3-transferase and a membrane permeability that is 100-1000 times higher, and the said protein complex consequently ensures a much better intracellular availability. This is the reason why only small amounts of C3-C2IN are needed now to achieve a 90% inhibition of Rho A-C. The activity has so far been only demonstrated in in vitro experiments [see S. Wahl, H. Barth, T. Ciossek, K. Aktories and B. K. Mueller, J. Cell. Biol., 149 (2000), 263-270]. It has been possible to observe the neurite growth stimulating effects in these experiments, which were carried out in embryo cells or cell lines, i.e. in proliferation competent cells (see S. Wahl: Ph.D. Thesis in Natural Sciences, Faculty of Biology, Eberhard-Karls University of Tübingen, 2000), The neurite treated with C3-C2IN and C2II were resistant to powerful inhibitors like ephrin A5 and RGM [see S. Wahl 2000, quoted above].

In addition, a second chimeric fusion protein—a chimeric C. botulinum C2/C3 inhibitor—has also been described (see WO Patent No. 99/08533). In the case of this chimeric product, the domain of C2 that possesses the ADP-ribosylating activity is deleted and replaced by the C3 enzyme. The result is therefore a C2II-C3fusion protein.

However, no application has so far been known with which it would be possible to achieve a lasting in vivo stimulation of adult nerve cells, i.e. cells with greatly restricted proliferative properties. The in vivo use of C3 has only been possible on freshly severed nerve cells, and it only led to short-term effects, since C3 is only taken up by the regenerating nerve fibers to a small extent [see M. Lehmann, A. Fournier, I. Selles-Navarro, P. Dergham, A. Sebok, N. Leclerc, G. Tigyi and I. McKerracher, J. Neurosci., 19 (1999) 7537-7547]. For this reason, and because of the large doses one would need to use, this system is not suitable for restoring the motor or sensory function after the spinal cord has been severed. No in vivo data are available for other Rho inhibitors. The aim of the present invention is therefore to achieve an in vivo restoration of the function of nerve fibers after injury or in the course of diseases and so help them regain their functions. The aim is therefore to obtain a full or partial regeneration in the case of diseases or injuries of the peripheral and central nervous system.

The present invention therefore relates to the use of a composition that contains a fusion protein and at least one transporter, for the in vivo stimulation of nerve growth, the in vivo inhibition of scar tissue formation, the in vivo reduction of secondary damage, and/or the accumulation of macrophages, where the fusion protein contains at least one binding domain for the transporter and at least one modulation domain for the covalent modification of small GTP-binding proteins, and where the transporter ensures the uptake of the fusion protein in the target cell.

When the said composition is used in accordance with the invention, surprisingly not only the effects of the myelin-associated inhibitors such as NOGO, MAG and CSPG are abolished, but also the effects of other powerful inhibitors, such as for example semaphorin and the repulsive guidance molecule (RGM), as well as the inhibitory cicatrix-associated chondroitin sulfate proteoglycans.

When using the system described above, surprisingly not only the blockade of the neurite growth by regeneration-inhibiting proteins can be abolished, but also the nerve fiber growth can be actively stimulated.

It was also surprising to find that the use of the composition according to the invention not only effected the inactivation of Rho A-C, but it also brought about the activation of Cdc42 and Rac. The activation of Rac and Cdc42 in nerve cells leads to the formation of fingerlike filopodia and lamellipodia (pellicles between the filopodia) see [R. Kozma, S. Sarner, S. Ahmed and L. Lim, Mol. Cell Biol., 17 (1997), 1201-1211]. Filopodia and lamellipodia are necessary for the target-oriented growth of nerve fibers. However, the activators of Rho, such as for example the myelin inhibitors RGM or ephrin A5, which attack the nerve fibers from the outside, inhibit the further development of the nerve fibers by causing a drastic retraction of the nerve fibers. The inhibition of Rho A-C stops this, but it does not lead by itself to the further growth of the nerve fibers, since this calls for the activation of Cdc42 and Rac. It is therefore the combination of the inhibition of Rho A-C and the activation of Cdc42 and Rac that is particularly efficient in stimulating the growth of nerve fibers.

Surprisingly, the number of ED-1 positive macrophages at the site of the injection greatly increased when the system described above was used. Macrophages remove the regeneration-inhibiting cell residues and the inhibitory myelin constituents from the site of injury, and they secrete cytokines, which modulate the activity of astrocytes and oligodendrocytes and so promote the regeneration of nerve fibers. Furthermore, the macrophages, which soon appear at the site of the injury induce the new remyelinization of demyelinized nerve fibers [see M. R. Kotter, A. Setzu, F. J. Sim, N. van Rooijen and R. J. M. Franklin, Glia, 35 (2001) 204-212].

It was also surprising to observe that, when using the composition according to the invention, the number of cicatrix-forming astrocytes was reduced, and so the formation of scar tissue also diminished. Less scar tissue was formed, and the scar tissue grew in a less compact manner, so that there was enough room left for the growth of nerve fibers. The formation of lacunae and cavities was much less pronounced, and so the secondary damage was drastically reduced. This had a favorable effect on the regeneration of nerve fibers.

All the effects together achieved, after a lesion to the spinal cord, the restoration of the motor, sensory and vegetative functions over the site of the spinal cord injury. In particular, it was possible to show that rats with a lesion to their spinal cord affecting the eighth thoracic vertebra (TH8) could again move their hind legs and did not retain any notable symptoms of paralysis after the administration of a single injection of C3-C2IN and C2II into their hind legs. Besides the motor function, the sensory and vegetative functions were also restored. The rats reacted to external stimuli (e.g. pain) and were able to empty their bladder unaided (see Example 2).

In the context of the present invention, the in vivo stimulation of nerve growth means an accelerated and/or improved nerve growth, which can apply to the extent and/or the speed of growth. The nerve fibers preferably grow about twice, especially about three times and more especially about four times faster and/or further. Alternatively, the number of growing fibers is increased at least by a factor of about 2, preferably by a factor or about 3 and more especially by a factor of about 4. In the context of the present invention, the in vivo inhibition of scar tissue formation means an approximately 50%, preferably an approximately 75% and more especially an approximately 90% reduction in the scar tissue and/or in the formation of lacunae and cavities. This indicates that the inhibition can be either full or partial. Suitable tests for quantifying the parameters are described in Example 2. Secondary damage, as opposed to primary damage, means damage that occurs as a sequela to the initial injury (the primary damage). In the context of the present invention, secondary damage, as opposed to primary damage, is the enlargement of the site of the initial lesion (the primary damage), caused by pathophysiologic mechanisms.

Examples of this are ischemia necrosis and the apoptosis of nerve fibers and other cells, as well as inflammatory reactions. In the context of the present invention, the in vivo reduction of secondary damage means an approximately 50%, preferably approximately 75% and more especially an approximately 90% reduction of secondary damage. The accumulation of macrophages means an increase in the number of macrophages, especially at the site of action and/or administration. The number of macrophages increases by a factor of at least about 2, preferably by a factor of about 3 and more especially by a factor of about 4. The site of action is the site where the composition according to the invention exerts its effect on the neurons, the nerve tissues and/or the adjacent cells or tissues. In the context of the present invention, the site of administration is the site where the composition according to the invention is released into the body. The fusion protein is an expression product of a fused gene. A fused gene is formed by the coupling of two or more genes or gene fragments, giving rise to a new combination. In the present invention, the fusion protein contains a modulation domain and a binding domain.

A GTP-binding protein is a protein that binds guanosine, triphosphate (GTP) and hydrolyzes it to guanosine diphosphate (GDP), owing to a cellular signal cascade. The signal-induced hydrolysis of GTP to GDP brings about the interaction between the GTF-binding protein and an effector molecule. We distinguish between heterotrimeric (or large) GTP-binding proteins and monomeric (or small) GTP-binding proteins. The heterotrimeric GTP-binding proteins consist of an α-, a β- and a γ-subunit, while the monomeric GTP-binding proteins only consist of a single subunit. The group of small GTP-binding proteins comprises for example the members of the Ras, Rho, Rab, Arf, Sar and Ran groups. The mammalian Rho GTPases can be divided into six classes: Rho (Rho A, Rho B, Rho C), Rac (Rac 1, Rac 2, Rac 3, Rho G), Cdc42 (Cdc42Hs, G25K, TC10), Rnd (RhoE/Rnd3, Rnd11/Rho6, Rnd2/Rnd7), Rho D and TTF.

A GTP-binding protein has a guanine nucleotide-binding site, which can bind both GTP and GDP. The protein is active in the GTP-bound form but inactive in the GDP-bound form. The exchange of GDP and GTP and so the activation of the GTP-binding molecule is mediated by the activator that occurs upstream of the GTP-binding protein in the signal cascade. As a result of the activation of the effector, i.e. of the molecule that occurs downstream of the GTP-binding protein in the signal transduction, GTP is split into GDP and inorganic phosphate. This again inactivates the GTP-binding protein. The regulation of the signal cascade at the level of the GTP-binding protein is further regulated, in thee cells by at least three further proteins: GTPase-activating proteins (GAP), which support the GTP hydrolysis, guanine nucleotide exchange factors (GEF), which catalyze the exchange of GDP for GTP, and GDP dissociation inhibitors (GDI), which suppress the dissociation of GDP by the small GTP-binding protein [see A. Hall, Science, 279 (1998) 509-514; also B. K. Mueller, Annu. Rev. Neurosci., 22 (1999), 351-388; and L. Luo, Nature Review Neurosci., 1,3 (2000), 173-180].

When the composition according to the invention is used, the activity of the small GTP-binding protein is altered. In the context of the present invention, the change of activity of the small, GTP-binding proteins means either a rise or a drop in activity. The drop in activity can mean a full or partial inhibition or inactivation. The activity of the small GTP-binding protein is raised or lowered by a factor of at least 2, preferably by a factor of about 3 or about 4, and more especially by a factor of about 10. The expert in the field is familiar with the relevant method used for determining the activity of small GTP-binding proteins. For example, it is possible to conduct an enzymatic test to determine the hydrolytic activity of the small GTP-binding protein, using GTP as the substrate that has been labelled e.g. radioactively on the γ-phosphate group [see P. W. Read and R. K. Nakamoto, Methods in Enzymology, 325 (2000), 15 and A. J. Self and A. Hall, Methods in Enzymology, 256 (1995), 67].

The change in activity due to the modulation domain can be brought about for example by interaction with GAP, GDI, GEF or the small GTP-binding protein. This can influence for example the rate of the hydrolysis of GTP to GDP, the dissociation of GDP, or the binding of GTP. This can be achieved for example by the covalent or non-covalent modification of one of the participating proteins by the modulation domain [see A. L. Bishop and A. Hall: “Rho GTPases and their effector proteins”, Biochem. J., 348(2000), 241-255; also A. Hall, “Signal transduction pathways regulated by the Rho family of small GTPases”, Br. J. Cancer, (80 Suppl,), I (1999) 25-27; and L. Kjoller and A. Hall “Signaling to Rho GTPases”, Exp. Cell res., 253 (1999), 166-179].

In a preferred embodiment, the small GTP-binding molecule—preferably Rho A-C—is fully or partly inhibited by covalent modification. This is preferably the result of the ADP-ribosylation or glycosylation of the small GTP-binding protein, that is to say, ADP-ribose or a saccharide is bound covalently. This modification leads to a changed signal transduction at the level of the small GTP-binding molecule.

In another embodiment, the change in the activity of the small GTP-binding protein is obtained by a non-covalent modification. For example, a molecule could be added onto the small GTP-binding protein, this molecule stabilizing an active or inactive form e.g. by altering the conformation of the protein. In another embodiment, however, a molecule could also be intercalated in the binding region of the small GTP-binding protein, so that GTP cannot be bound any more and therefore the activity of the small GTP-binding protein is reduced. For example, the activity of Rho GTPase can be altered by Rho-inhibiting toxins such as e.g. Exos (Pseudomonas aeruginosa exoenezyme S), SptP (Salmonella typhimurium protein tyrosine phosphatase) or YopE (Yersinia pseudotuberculosis outer protein E), or else by Rho-activating toxins such as e.g. SopE. (Salmonella typhimurium outer protein E [see M. Lerm, G. Schmidt and K. Aktories, FEMS Microbiology Letters 188 (2000), 1-6; and K. Aktories, G. Schmidt and I. Just, Biol. Chem., 381 (2000), 421-426].

In another embodiment the modification is brought about not by the modulation domain itself but by a signal molecule that is located either upstream or downstream of the small GTP-binding protein in the signal cascade. The modulation domain would then activate such a signal molecule, which in turn e.g. phosphorylates the small GTP-binding protein (indirect modulation). For example, protein kinase A (PKA) phosphorylates GTP-bound Rho A that is active in the lymphocytes, and it induces its translocation from the membrane to the cytosol via Rho-GDI, as a result of which the Rho activation is terminated in two ways. The signal molecule cAMP activates PKA, this phosphorylates RhoA and consequently inhibits it, while at the same time the activation of RhoA is inhibited by the transport of Rho-GDI from the membrane to the cytosol [see B. K. Mueller, Annu. Rev. Neurosci., ZZ (1999), 351-388; also P. Lang, F. Gespert, M. Delespine-Carmagnat, R. Stancou, M. Pouchelet and J. Bertoglio, EMBO J., 15 (1996, 510-519; and C. Laudanna, J. J. Campbell and E. C. Butcher, J. Biol. Chem, 272 (1997), 24, 141-24,144].

In a particularly preferred embodiment, the small GTP-binding proteins Rho A, B or C are modified covalently. The ADP-ribosylation of the aspartic acid group in position 41 is especially preferred. This causes the inactivation of the small GTP-binding protein. In another embodiment, the threonine group in position 35 or 37 of a small GTP-binding protein of the Rho family is glycosylated. This also leads to the inactivation of the small GTP-binding protein. At the same time, preferably the small GTP-binding proteins Cdc42 and/or Rac are activated. This might happen for example by “crosstalk” between the two signal transduction pathways. The expert in the field uses the term “crosstalk” to denote the mutual influence between various signal transduction pathways within the same cell. In the present case, the inactivation of the signal pathway that comprises the GTP-binding proteins Rho A, B, or C for example can bring about the activation of the signal pathway involving Cdc42 and/or Rac [see in this connection B. K. Mueller, Annu. Rev. Neurosci., 22 (1999), 351-388; also S. Wahl, H. Barth, T. Ciossek, K. Aktories and B. K. Mueller, J. Cell. Biol., 149 (2000), 263-270; and E. E. Sander, J. P. Ten Klooster, S. Van Delft, R. A. Van der Kammen and J. G. Collard, J. Cell Biol., 147 (1999), 1009-1022].

The modulation domain is preferably derived, from a toxin. This can be e.g. a bacterial toxin. Bacterial toxins can be obtained from the genera Clostridium, Staphylococcus, Bacillus, Pseudomonas, Salmonella or Yersinia. In a preferred embodiment, the C3-transferase from Clostridium botulinum or a related transferase is used. “Related transferase” means an enzyme that—similarly to C3-transferase—brings about the ADP-ribosylation of GTP binding proteins belonging to the Rho family.

The binding domain is another part of the fusion protein. It brings about binding to the transporter. The binding of the binding domain to the transporter occurs e.g. by a covalent bond, electrostatic interactions, Van der Waals forces or hydrogen bonds. In one embodiment, the binding domain is derived from a binary bacterial toxin, especially C2 toxin obtained from Clostridium botulinum.

The term “binary toxin” means a toxin that consists of two separate proteins. The enzyme component and the cell binding and translocation component are both proteins. Examples of binary toxins are the anthrax toxin and the toxin obtained from Clostridium perfringens iota. The Clostridium perfringens iota toxin is a member of the group of binary actin ADP-ribosylating toxins. In a particularly preferred case the binding domain is derived from the C2 toxin obtained from Clostridium botulinum. In the most favourable embodiment, the binding domain is the N-terminal C21 domain of the C2 toxin from Clostridium botulinum.

The transporter effects the uptake of the fusion protein in the cell. The transporter can be for example a peptide or a protein. An example of such a protein or peptide is the antennapedia peptide, which is a peptide that is built up of 16 amino acids and which belongs to the homeobox gene antennapedia. This is used to insert exogenous hydrophilic components into the living cell [see A. Prochiantz, Ann. N.Y. Acad, Sci., 866 (1999), 172-179; also A. Prochiantz, Curr. Opin. Neurobiol., 6 (1996), 629-634].

However, the transporter can also be a viral protein or a ligand for a cell surface structure, or it can be derived therefrom. An example of a viral transport protein is VP22, a large structural protein of Herpes simplex virus 1 with 38 kDA. This protein translocates the plasma membranes of mammalian cells and can act as a transporter for transferring other proteins into the cells [see P. O'Hare and G. Elliot, Cell, 88, (1997), 223-233 also A. Phelan, G. Eliott and P. O'Hare, Nat. Biotechnol., 16 (1998), 440-443]. The plant toxin ricin and the bacterial shiga toxin are examples of ligands of surface structures [see K. Sandvig and B. van Deurs, EMBO J., 19 (2000), 5943-5950].

However, for example liposomes can also fulfill the transport function. When liposomal transporters are used, not only nucleic acids but also proteins can be introduced into cells [see M. Rao and C. R. Alving, Adv. Drug Delv. Res., 30, (2000), 171-188]. The uptake in the cells can occur for example by fusion through the cell membrane, by the crossing of the cell pores, by facilitated diffusion, by active transport with the aid of the carrier in the cell membrane, or by pinocytosis and phagocytosis. In one of the embodiments, the uptake of the fusion protein occurs via the binding of the transporter to a structure on the cell surface. This structure can be e.g. a receptor, a channel or another membrane protein. The structure on the surface ensures the uptake of the composition or a part of it in the cell. The uptake of the fusion protein can occur e.g. via the endocytosis of a receptor-protein complex. The protein complex can be released in the cell and then it can alter the activity of the small GTP-binding protein.

The transporter can also be e.g. a ligand. Ligands are molecules that bind specifically on certain receptors. These ligands can be for example physiologic molecules like hormones, neurotransmitters like e.g. acetylcholine, or nonphysiologic molecules like artificially prepared ligands. The ligands can be of peptidergic, proteinergic or non-proteinergic origin. In one of the embodiments, the transporter can represent the variable region of an antibody, e.g. a monoclonal antibody, or else it can be combined therewith. This region could ensure the specific binding on the cell surface structures.

However, the uptake in the cell can also be effected by liposome transporters. [see M. Rao and C. R. Alving, Adv. Drug Delv. Res., 30 (2000), 171-188]. In this case, the fusion protein would be surrounded e.g. by liposomes. The binding domain would be so formed as to make the fusion protein particularly suitable for enclosure in a liposome. The liposome would fuse with the cell membrane and so effect the uptake of the fusion protein in the cell. The expert in the field is familiar with suitable lipids that can be used to form protein-liposome complexes.

Another possibility would be the uptake of the fusion protein with the aid of a viral transporter. An example of viral transporters is the above-mentioned VP22 [see P. O'Hare and C. Elliot, Cell, 88 (1997), 223-233; also A. Phelan, G. Elliott and P. O'Hare, Nat. Biotechnol., 16 (1998), 440-443].

In a preferred embodiment the transporter is derived from a binary bacterial toxin. Examples of binary toxins are the anthrax toxin and the toxin obtained from Clostridium perfringens iota. The Clostridium perfringens iota toxin is a member of the group of binary actin-ADP-ribosylating toxins. In a particularly preferred case, the transporter is derived from the C2 toxin from Clostridium botulinum. In the most preferred embodiment, the transporter protein is the C2II domain of the C2 toxin obtained from Clostridium botulinum.

The medicinal product comprising the composition that contains at least one fusion protein and at least one transporter is prepared in the usual way, using the current processes of pharmaceutical technology. For this purpose, the active substances are included as such or in the form of their salts, together with suitable pharmaceutically acceptable excipients and additives, in order to obtain pharmaceutical forms that are suitable for the indication and for the method of application.

The expert in the field is familiar with the suitable excipients and additives, which serve for example the purpose of stabilizing or preserving the medicinal product or are used as diagnostic aids [see e.g. H. Sucker et. al., “Pharmazeutische Technologie” (=Pharmaceutical Technology), 2nd ed., Georg Thieme Verlag, Stuttgart, Germany, 1991]. Examples of such excipients and/or additives are antimicrobial compounds, proteinase inhibitors, sterilized water, pH-adjusting substances such as e.g. organic and inorganic acids and bases, as well as salts thereof, buffering substances for adjusting the pH, substances used to make the preparation isotonic, such as e.g. sodium chloride, sodium bicarbonate, glucose and fructose, surfactants or surface-active substances and emulsifiers such as e.g. the partial fatty acid esters of polyoxyethylene sorbitan (Tween®) or e.g. the fatty acid esters of polyoxyethylene (Cremophor®), fatty oils such as e.g. peanut oil, soybean oil and castor oil, synthetic fatty acid esters such as e.g. ethyl oleate, isopropyl myristate and neutral oil (Miglyol®), as well as polymeric excipients such as e.g. gelatin, dextran, polyvinylpyrrolidone, solubililizing agents, organic solvents such as e.g. propylene glycol, ethanol, N,N-dimethylacetamide and propylene glycol, complexants such as e.g. citrates and urea, preservatives like e.g. hydroxypropyl benzoate and methyl benzoate, benzyl alcohol, antioxidants such as e.g. sodium sulfite, and stabilizers such as e.g. EDTA.

The medicinal product can be in a form suitable for parenteral administration and especially in a form suitable for intrathecal, intramedullary, intraarterial, intravenous, intramuscular or subcutaneous application, especially at the site of the injury. It can also be in a form suitable for intradermal application, for example as plasters (patches), enteric application, especially for oral or rectal use, or topical application, especially as a cutaneous preparation.

The terms “acute injury” and “acute brain and/or spinal cord disease” are used here in contradistinction to “chronic disease” to mean an injury or disease that occurs suddenly. Examples of these are: skull and brain injuries caused by an external traumatic event, infections caused by bacterial viruses, fungi and parasites; stroke (cerebral circulatory disturbance and intracerebral or subarachnoid haemorrhage); intoxications: and traumatic lesions of the spinal cord.

The term “chronic injury and/or diseases of the brain or the spinal cord” is used here to mean a disease that has a slow, insidious onset and generally a long duration. Examples of chronic diseases of the brain and the spinal cord are Alzheimer's disease, Parkinson's disease, multiple sclerosis, tumors and similar diseases.

Multiple sclerosis and leukodystrophy are examples of inflammatory diseases of the nervous system, which are accompanied by demyelinizing damage.

The term “remyelinization” is used here to mean the complete or partial restoration of the myelin layer after demyelinization. Demyelinization is damage to and/or loss of the myelin in the central or peripheral nervous system; it can arise as a result of various diseases of the nervous system or after general damage to the neurons or the oligodendrocytes, caused for example by inflammatory, immunopathologic or toxic processes. Examples of this are multiple sclerosis, leukodystrophy and viral diseases, like canine distemper.

The term “neurologic and neurodegenerative diseases of the peripheral and central nervous system” is used here to cover for example Alzheimer's disease, Parkinson's disease, multiple sclerosis and similar diseases that are accompanied by a lose of nerve fibers and by demyelinization, together with amyotrophic lateral sclerosis and other motor neuron diseases, as well as ischemia, stroke, epilepsy, Huntington's disease, the AIDS-dementia complex, and prion diseases.

The illustrations and examples that follow are used to explain the invention in more detail but the invention is not limited to them.

### DESCRIPTION OF THE FIGURES

FIG. 1—Schematic representation of the particularly preferred system containing one fusion protein and one transporter protein

The fusion protein consists of a modulation domain, which is derived from C3-transferase obtained from C. limosum, and of a binding domain, which is derived from the N-terminal end of the C2I subunit of the C2 toxin obtained from C. botulinium. The transporter is the C2II, subunit of the C2 toxin from C. botulinum.

The individual parts of the figure represent the following molecules:

A=C2 toxin from Clostridium botulinum

B=C3 exoenzyme from Clostridium limosum

C=C2II transporter protein

D=C3-C2IN fusion protein consisting of the binding domain (C2IN) and the modulation domain (C3).

FIG. 2—Improvement of the motor functions after the administration of C3-C2IN

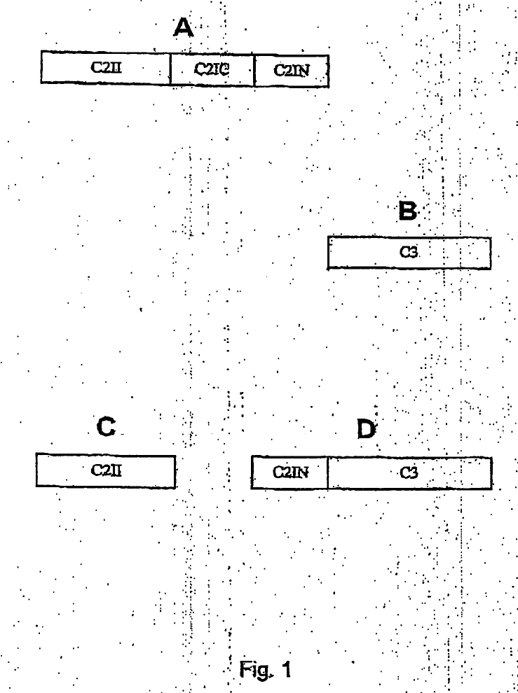

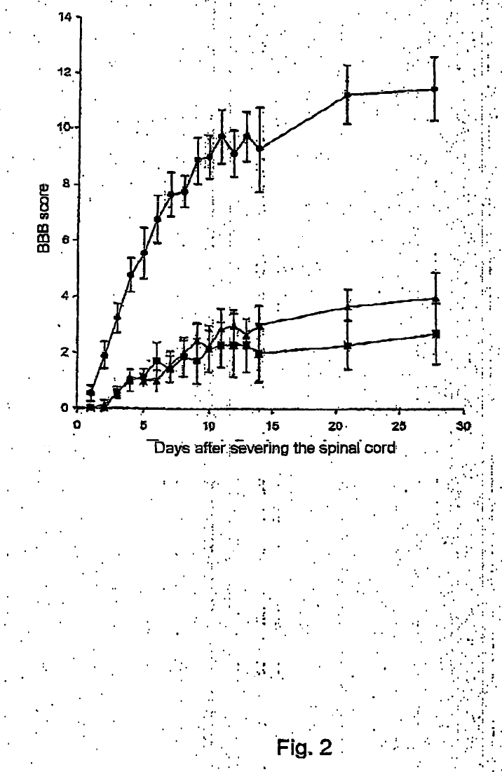

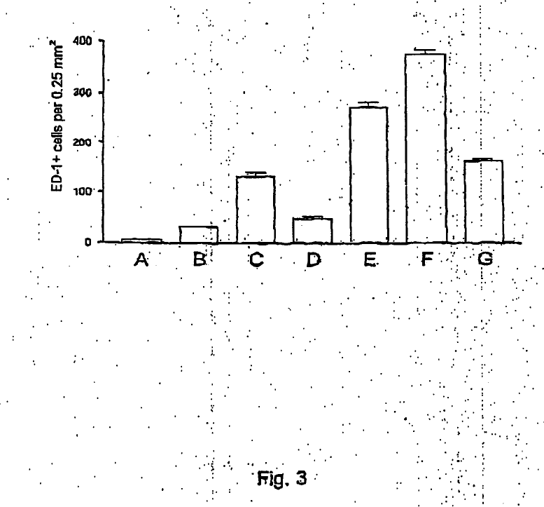

The restoration of the motor function in differently treated animals was determined as a function of the recovery time. The rats treated with C3-C2IN in the presence of C2II, marked with a circle (●) showed a considerably better motor recovery than the control animals, marked with a triangle (▴), and the animals that were treated only with C2II, marked with a square (▪). After 28 days, the animals treated with C3-C2IN reached a value of 11.50 (±1.15) on the BBB scale, while the control animals and the animals treated only with C2II had a value of only 4.00 (±0.90) and 2.71 (±1.09), respectively.

FIG. 3—Intraspinal accumulation of activated macrophages after the administration of C3-C2IN/C2II The number of ED-1 positive macrophages, arising in response to the intramedullary injection of 10 μg of C3-C2IN and 10 μg of C2II was determined after 1, 3 and 7 days, as well as after 4 weeks. For a control, the number of ED-1 positive macrophages was determined on days 1 and 3 in animals that had not received any substance and in animals that had received phosphate buffered saline (PBS) at a pH of 7.4. After only one day following the injection of the substances, the number of macrophages was already higher by a factor of 23, and on day 3 it was higher by a factor of 47. The number of macrophages reached the maximum on the seventh day (increase by a factor of 65), and after 4 weeks, the number was 162 ED-1 positive macrophages per 0.25 mm², which was still 28 times higher than the normal value of 5.8 ED-1 positive macrophages per 6.25 mm².

The individual columns in the figure have the following meanings:

A=untreated

B=given PBS determination on day 1

C=treated with C3-C2IN/C2II determination on day 1

D=given PBS determination on day 3

E=given C3-C2IN/C2II determination on day 3

F=given C3-C2IN/C2II determination on day 7

G=given C3-C2IN/C2II determination after 4 weeks.

FIG. 4—Histologolic picture of the intraspinal accumulation of activated macrophages after the administration of C3/C2IN/C2II

There was a great increase in the accumulation of ED-1 positive macrophages in response to the intramedullary injection of 10 μg of C3-C2IN and 10 μg of C2II directly at the site of the injection. The immunohistologic staining of the macrophage 1 cm from the site of injection still indicated an enhanced accumulation in comparison with the controls.

The individual parts of the figure have the following meanings:

A=ED-1 positive macrophages at the site of the injection of C3-C2IN/C2II

B=ED-1 positive macrophages at a point 1 cm from the site of injection of C3-C2IN/C2II

C=Controls/untreated.

FIG. 5—Reduced intraspinal accumulation of vimentin⁺ reactive astrocytes and fibroblastoid cells after the administration of C3-C2IN/C2II

Activated astrocytes and fibroblastoid cells were immunochemically marked with vimentin antibodies and then counted. The number of vimentin-positive reactive astrocytes and fibroblastoid cells 3 days after the administration of C3-C2IN/C2II (C) was greatly reduced in comparison with the case when only PBS was administered (B). (A) shows the number of vimentin-positive reactive astrocytes and fibroblastoid cells in the untreated control animals.

The individual parts of the figure show the number of vimentin⁺ reactive astrocytes and fibroblastoid cells in the following animals:

A=untreated animals

B=animals treated with PBS

C=animals treated with C3-C2IN/C2II.

FIG. 6—Growth assay of retinal ganglion cell axons on chondroitin sulfate protoglycan (CSPG)

The neutralization of the inhibitory scar tissue parts by C3-C2IN/C2II was demonstrated by the growth test carried out on CSPG. For this purpose, retina mini-explantates from chick embryos (E7) were plated out on cover glasses coated with 20 μg of CSPG per ml. CSPG inhibited the growth of the retinal ganglion cell axons (A). The addition of 1 ml of C3-C2IN/C2II (300 ng/ml) led to the neutralization of the inhibitory effect of CSPG and to the growth of retinal axons (B).

The individual parts of the figure show the growth of retinal ganglion cell axons in the case of:

A=incubation with CSPG

B=incubation with CSPG and C3-C2IN/C2II.

### EXAMPLE 1

The proteins were constructed, expressed, purified and characterized as described in Examples 1-5 in German Patent No. 197 35 105 A1.

### EXAMPLE 2

Animals

Male Lewis rats aged 8-12 weeks and weighing 220-280 g (from Charles River, Sulfeld, Germany) were randomly divided into two groups, and their spinal cord was at least half severed. After 21 days, the animals in one group were infused with 10 μg of C3-CC2IN and the animals in the other group were infused only with 10 μg of C2II. The control animals received either 10 μg of C2 alone (without the C3 component) or had a transection without injection. All the animals were kept under conditions of controlled light and temperature and received food and water ad libitum. The rats were kept in accordance with the International Health Guidelines and in accordance with a protocol checked out by the University of Tübingen.

The rats were anesthetized by the intraperitoneal injection of ketamime hydrochloride (Ketanest, from Parke Davis, 100 mg/kg) and xylazine hydrochloride (Rompun, from Bayer, 10 mg/kg). To prevent the drying out of the eyes during anesthesia both eyes were covered with retinol palmitate (Oculotect Gel, from CIBA Vision, Novartis, Germany). When a sufficient level of anesthesia had been reached, the skin over the vertebral column was incised, the muscles attached to the vertebrae were separated, and the spinal cord was released by bilaminectomy at the level of the eighth thoracic segment (TH8). After opening the dura mater (the outermost fibrous envelope of the spinal cord), the dorsal spinal tract was cut through two-thirds of the way (i.e. more than by a hemisection), using a pair of fine iridectomy scissors. The severed neural structures were both of the motor type (the crossed part of the pyramidal tract and parts of the extrapyramidal tract) and of the sensory type (dorsal spinal cord). The wound was rinsed with sterile saline and closed. All the animals were warmed under an infrared lamp until they regained consciousness.

Postoperative Treatment and Tissue Preparation

All the animals received a postoperative analgesic treatment in the form of a single intraperitoneal injection of Rimadyl in a dose of 2 mg/kg (Carproven, from Pfizer, Germany), and their bladder was emptied manually three times a day until their spontaneous bladder function was reestablished (generally within 10-14 days). Prior to the reestablishment of the spontaneous bladder function, the rats were bathed twice or three times a day in order to prevent wounds being caused by urine. The animals were regularly weighed, and in the case of a weight loss of 20% or more they were sacrificed. For the immunohistologic examination, the rats were sacrificed and infused intracardially with the fixative, which was a 4% formalin solution in 0.1 mole/l of phosphate buffer at pH 7.5, and which contained 20,000 IU of heparin per liter. The spinal cord and the brain were removed and fixed again overnight at 4° C. The fixed tissues were embedded in paraffin. Serial sections were prepared and transferred to microscope slides coated with silane.

Immunohistochemistry

After mixing the material in formalin and embedding it in paraffin, rehydrated pieces measuring 2 μm were boiled seven times for 5 minutes in citrate buffer (2.1 g/l of sodium citrate at pH 6) and incubated with 10% of normal hog serum (from Biochrom, Berlin, Germany) in order to suppress the a specific binding of immunoglobulins. Antibodies to cell-specific antigens were used to identify the various cell types. These included glial fibrillary acidic protein (GFAP) from Boehringer Mannheim, Germany, 1:100) for astrocytes, myelin basic protein (MBP from Dako, Glostrup, Denmark, 1:200) for oligodendrocytes, and neurofilament (from Dako, Glostrup, Denmark, 1: 200) for neurons. The microglia and macrophages were marked with monoclonal antibodies to ED1 (from Serotec, Oxford, Great Britain; 1: 100), OX-42 (from Serotex, Oxford, GB, 1: 100) or ED2 (from Serotec, Oxford, GB). These were used in conjunction with the ABC process (avidin-biotin complex) in combination with alkaline phosphatase conjugates. In addition, we used monoclonal antibodies to OX-22 (from Serotec, Oxford, Great Britain, 1:100) for the identification of T lymphocytes, and W3/13 (from Serotec, Oxford, Great Britain, 1: 100) for the identification of T lymphocytes. OX-6 (from Serotec, Oxford, Great Britain 1: 100) was used for the identification of MHC-II molecules in order to characterize the functional immunocompetence. The antibodies were placed on the microscope slides in the solutions mentioned above, using bovine serum albumin buffered with 1% of tris (BSA/TBS). The binding was visualized by the addition of a biotin-coupled second antibody (1:400, 30 min.) and an alkaline phosphatase conjugated ABC complex (1: 400 in BSA/TBS; 30 min.).

Histologic Staining Myelin and Nuclein

The serial tissue sections used for the immunohistochemical investigations were stained with Luxol fast blue for myelin. The tissue areas that were evidently damaged or showed a deficiency of myelin were identified, starting at the center of the lesion and proceeding in the rostral and caudal direction in steps, to points at various distances (0.6, 1.2, 1.8, 2.4 and 3.0 cm). The nuclei were stained with cresyl violet (0.1%) in order to be able to distinguish intact and damaged areas in the gray substance. The thin sections showed that the secondary damage was less pronounced in the rats treated with C3-C2IN in the presence of C2II. The formation of lacunae and cavities was markedly less in the treated animals than in the controls. At the same time, more cells, fewer recesses and an increased neuron proliferation could be observed.

Stereotactic Micro-injection

Microcapillaries and a stereotactic apparatus were used to inject exact amounts (10×1 μl, 10 μg) of C3-C2IN toxin into the rostral stump of the severed spinal cord. To stabilize the spinal cord further, a device was built for lifting the rats up, which blocked the extension of the respiratory movement to the vertebral column.

Anterograde Marking

Biotinylated biodextran (from BDA, with 10,000 kDa) was injected in an amount of 30 μl (30 μg, 15 μl per side) into the motor cortex region with the aid of a Hamilton syringe. After the injection the wound was washed and closed. The aim of this method was to demonstrate the regenerated axon fibers in the corticospinal tract (CST). The biotinylated biodextran is transported from the motor cortex region to the spinal cord. All the fibers that contained biotinylated biodextran under the lesion area must therefore be newly formed. The rats treated with C3-C2IN in the presence of C2II showed a markedly greater nerve fiber growth than the control animals. Both the number of the fibers and the length of the newly grown fibers were markedly greater here. The newly grown fibers were GAP43 positive (observed with the aid of polyclonal antibodies), whereby they were identified as proliferating neuron fibers.

Sensory and Locomotor Assessment

The animals were observed for the recovery of their functional capacity over a period of 1-21 days after the injury and were assessed with the aid of the Combined Sensory-Motor Gale Score [see K. Gale, H. Kerasidis and J. R. Wrathhall, “Spinal cord contusion in the rat: behavioral analysis of functional neurologic impairment”, Exp. Neurol. 88 (1985), 123-134], using an inclined plane [see. A. S. Rivlin and C. H. Tator, “Objective clinical assessment of motor function after experimental spinal cord injury in the rat”, J. Neurosurg., 47 (1977), 577-581], as well as by the Motor Openfield BBB Score [see D. Basso, M. S. Beatti and J. C. Breshnahan, “Graded histological and locomotor outcomes after spinal cord contusion using the NYU weight-drop device versus transection”, Exp. Neurol., 139 (1996), 244-256].

In two independent experiments, rats that had received C3-C2IN showed a significant improvement (p<0.0001) in their sensory and motor function in comparison with the rats that had only received active or inactive C2transporter protein, or rats that belonged to the control group. The improvement in the sensory and motor function occurred already on the third day and reached its maximum 21 days after the injury. Prior to the application, the biological and functional activity of the C3-C2IN construct was tested in in vitro experiments concerning the collapse of the growth cone. The motor function was tested e.g. on toe spreading, bodily orientation, standing up, and by the inclined-plane test, while the sensory function was tested by noting the hind leg retraction reflex on pulling, pain (caused manually and by heat), pressure and by the swimming test. A third experiment was performed as a double-blind test and gave the same results.

In a fourth experiment, the relevance of the C2 transporter component to the functional recovery was analysed. The micro-injection of C3-C2IN and the inactive C2 component did not lead to a significant restoration of the function. These results show that the active transporter protein C2 is necessary for regeneration.

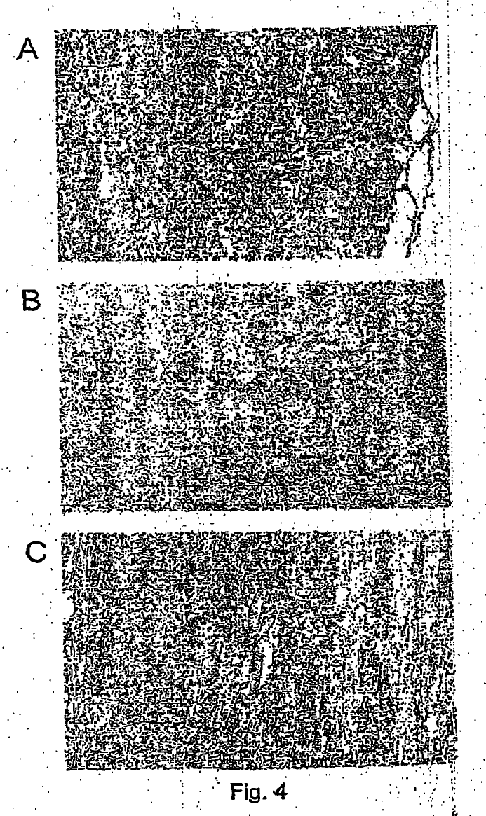

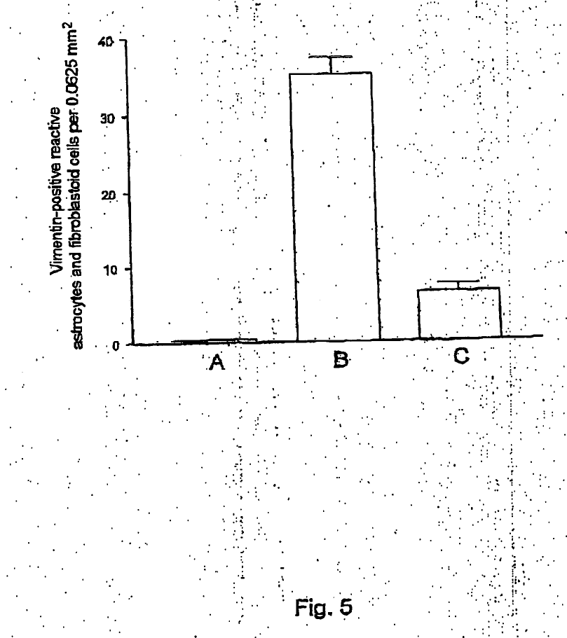

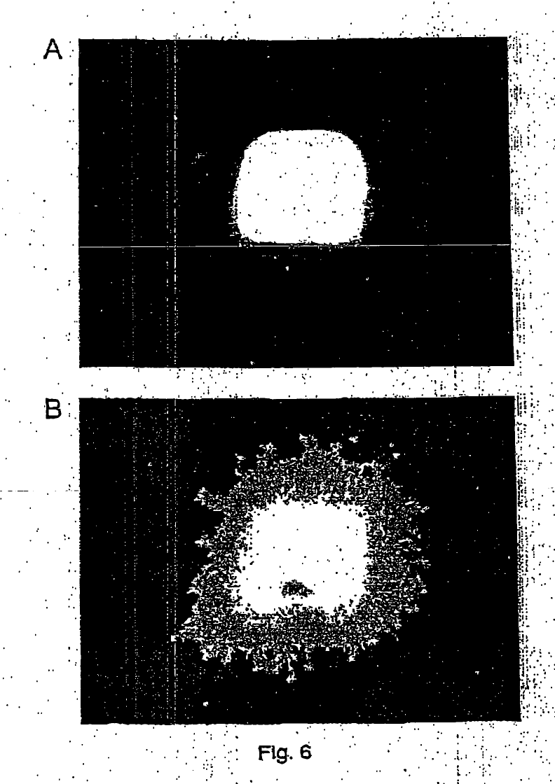

The improvement of the motor function in animals treated with C3-C2IN was shown in the appearance of a more functional holding of the hind legs (usually first bending at the hips then at the knees and finally the dorsiflexion of the ankles), which reached a maximum (average ±SEM) of 12.2 points (±0.84) in the BBB assessment (0-21 points), including weight bearing on the hind legs (see FIG. 2). The healing was symmetrical in most cases (<80%. The control animals reached a maximum of 4.1 points (±0.5) in the BBB assessment and did not show any progressive improvement during the period of observation. This agrees with the results obtained by other groups (see Basso et al, 1996, quoted above). On the tenth day after the severing operation, the animals receiving C3-C2IN showed an improvement of up to 8-9 points (“sweeping”) in comparison with that obtained at the time of the first examination, while the control animals only showed an improvement of less than 3 points. It was only in the first experiment that we observed an improvement of up to 8 points (±0.58) after 21 days also in the animals that had only received C2. In the second experiment, we could not confirm so far the effect of the sole administration of C2. Until the present day, a significant motor healing, including weight bearing on the hind legs, has been confined to rats that had received the C3-C2IN construct. No significant motor healing occurred in the animals treated with C3-C2IN and an inactive C2 transporter component, or with the C2 transporter component only, or without the addition of any component (control animals).

The sensory healing was quantified with the aid of Cales sensory motor assessment. Twenty-one days after the injection, rats treated with C3-C2IN retracted their hind legs in response to all the stimuli used (touching, mechanical reception and temperature) in a way that was comparable with untreated rats. The rats treated with C3-C2IN showed an up to 95% healing on the basis of this combined assessment, while rats treated with C2 and the control animals showed a healing of less than 50%. Furthermore, the animals treated with C3-C2IN exhibited a very pronounced tactile sense, which is essential for the specific holding of the hind legs.

The histological changes after the administration of C2-C3IN/C211 lie in the greatly increased number of ED-1 positive macrophages.

Morphological changes characterized by i) a reduced scar tissue formation and ii) reduced secondary damage, such as cavity formation, were observed in animals treated with C3-C2IN. The formation of new tissues was demonstrated by immunohistochemical methods (specific antibodies, nuclein staining), and the tissues spanning the site of the lesion were identified as tissues of neuron origin (neurofilament).

A retransection carried out above the first site of lesion (Gh7) caused a new paralysis of the hind quarters in animals that had shown a regeneration of more than 95%. The explanation of this is that the regeneration was in fact ensured by the corticospinal fibers above the first site of lesion, these fibers spanning the lesion.

### EXAMPLE 3

As in Example 2, a laminectomy was performed in rats at the level of the eighth thoracic segment (TH8), but the spinal cord was not severed subsequently. The dura mater was punctured 20 times in order to inject the spinal cord with doses of 10 μl of C3-C2I/C2II (2 μg per ml in PBS) or 10 of PBS.

As in Example 2, the animals were infused after three days, and the brain and spinal cord were removed and fixed. The tissues were then embedded in paraffin and cut into thin sections.

The vimentin reactive astrocytes and fibroblastoid cells were immunohistochemically marked with vimentin antibodies (from Dako, Glostrup, Denmark, 1:15).

The reduction in the formation of scar tissue in the animals treated with C3-C2I/C2II was evident from the histologic evaluation, which showed that the number of vimentin-positive astrocytes or fibroblastoid cells was less by a factor of 5.5 in comparison with that found in the animals treated with PBS.

### EXAMPLE 4

Growth Assay of Retinal Mini-explantates on CSPG

For this assay, we coated small cover glasses with poly-L-lycine (200 μg/l from Sigma, Germany for 30 minutes at room temperature) and with a protein mixture formed by CSPG (20 μg/ml, from Chemicon, Germany) and laminin (20 μg/ml, from Invitrogen, Germany) for 2 h at 37 C., after which they were washed with Hank's buffer (PAA, AT).

For the preparation of the retina mini-explantates, chick embryo eyes (E7) were removed, and the retina was isolated and placed flat on a tissue slicing plate, after which it was cut into squares measuring 150 μm×150 μm with the aid of a tissue cutter. The explantates were taken up in the culture medium (F12, PAA, AT; 10% of fetal calf serum Gold, PAA, AT; 2% of chicken serum, from Invitrogen, Germany; penicillin/streptomycin, 1:100, PAA, AT; glutamine, 1:100, PAA, AT), and 20-30 pieces were placed on the coated cover glasses with a pipet. These were cultured for 24 h at 37° C. in 4% of CO₂ on a plate containing 24 wells. 300 ng of C3-C2I/C32II were added at the time of explantation.

The mini-explantates were then fixed in 4% of PFA (from Merck, Germany) overnight at 4° C., and the cytoskeleton was visualized with the aid of phalloidin allexa stain (Allexa 488, from Molecular Probes, Holland), using the instructions.

CSPG inhibits the growth of the axons from the retinal mini-explantates. The addition of C3-C21/C2II neutralized this inhibitory action, and the axons grew out.

1. Use of a composition comprising at least one fusion protein and at least one transporter for the preparation of a medicinal product for the in vivo stimulation of nerve growth, the in vivo inhibition of scar tissue formation, the in vivo reduction of secondary damage and/or the in vivo accumulation of macrophages, where the fusion protein contains at least one binding domain for the transporter and at least one modulation domain for modifying the activity of small GTP-binding proteins, and where the transporter ensures the uptake of the fusion protein in a cell.

2. Use of a composition according to claim 1, characterized in that the transporter is bound to a structure on the surface of the cell, especially a receptor or a surface protein.

3. Use of a composition according to claim 1 or 2, characterized in that the transporter is a viral, liposomal, proteinergic or peptidergic transporter.

4. Use of a composition according to claims 1-3, characterized in that the transporter is derived from a binary bacterial toxin, especially C2 toxin, obtained from Clostridium botulinum.

5. Use of a composition according to claims 1-4, characterized in that the modulation domain inactivates the small GTP-binding proteins, especially Rho A-C.

6. Use of a composition according to claims 1-5, characterized in that the modulation domain inactivates the small GTP-binding proteins preferably by covalent modification and more especially by ADP ribosylation or glycosylation.

7. Use of a composition according to claims 1-4, characterized in that the modulation domain activates the small GTP-binding proteins, especially Cdc42 and Rac.

8. Use of a composition according to claims 1-7, characterized in that the modulation domain is derived from a toxin.

9. Use of a composition according to claim 8, characterized in that the toxin is a bacterial toxin, where the bacteria are chosen especially from the genera Clostridium, Staphylococcus, Bacillus, Pseudomonas, Salmonella or Yersinia.

10. Use of a composition according to claims 7 or 8, characterized in that the toxin is C3 exoenzyme obtained from Clostridium limosum or it is a related transferase.

11. Use of a composition according to claims 1-10, characterized in that the binding domain contains fully or partly a binary bacterial toxin, preferably C2 toxin obtained from Clostridium botulinum, and more especially the C2IN domain.

12. Use of a composition according to claims 1-11, for the treatment of neuron damage.

13. Use of a composition according to claims 1-12 for the treatment or an acute and/or chronic injury and/or disease of the brain and/or the spinal cord.

14. Use of a composition according to claims 1-13 the treatment of a neurologic and neurodegenerative disease of the central and/or peripheral nervous system.

15. Use of a composition according to claims 1-14 for the treatment of an inflammatory disease of the nervous system that is accompanied by demyelinization.

16. Use of a composition according to claims 1-15 for stimulating remyelinization.
